(19) United States
(12) Patent Application Publication
(10) Pub. No.: US 2016/0353782 A1
(43) Pub. Date: Dec. 8, 2016

(54) BEVERAGE COMPOSITION AND PROCESS OF PRODUCING THE SAME

(71) Applicant: Kurt Howard Ruppman, SR., Allen, TX (US)

(72) Inventor: Kurt Howard Ruppman, SR., Allen, TX (US)

(21) Appl. No.: 15/239,852

(22) Filed: Aug. 18, 2016

Related U.S. Application Data
(60) Provisional application No. 62/164,692, filed on May 21, 2015.

(51) Int. Cl. A23L 2/02; A61K 47/02; A61K 36/21; A23L 2/39; A61K 9/00

(52) U.S. Cl. CPC A23L 2/02; A23L 2/39; A61K 9/0095; A61K 36/21; A61K 47/02; A23V 2002/00

(57) ABSTRACT

The process of producing a beverage for enhancing athletic performance comprises selecting an amount of beet product as a solute. The process of producing a beverage includes infusing water with a molecular hydrogen in a desired amount to produce hydrogen infused water as a solvent. The process further includes mixing the solute into the solvent to produce the beverage for providing controlled reduction of nitrates. The proposed invention further relates to a beverage composition providing controlled reduction of nitrates in a human body. The composition of the beverage comprises one or more beet products, and a hydrogen infused water. The proposed beverage is capable of stabilizing the nitrates content, and controlling reduction of nitrates to nitric oxide in the blood plasma, which is effective for enhancing one's athletic performance.

### BACKGROUND

The present invention relates to a beverage composition and a process of producing the beverage using a beet product.

Nitric oxide is a gas composed of one molecule of nitrogen bonded to one molecule of oxygen. It serves as a critical signaling molecule in the cardiovascular and circulatory system in a human body. This essential molecule signals our blood vessels to relax and expand, creating greater efficiency in the body, greater nutrient delivery, and greater oxygenation to every system, organ and tissue in the body. The nitric oxide also helps in improving one's athletic performance.

Generally, diet and exercise play a large role in stimulating the natural production of nitric oxide. The nitric oxide stimulating foods or foods that are high in dietary nitrites/nitrates with regular exercise helps to promote nitric oxide production in the human body. Unfortunately, in today's world of refined foods these options may not be enough, especially for the athletes. Most of the athletes consume beverages consisting of functional foods that are high in dietary nitrites/nitrates such as spinach and beetroot.

The known art for preparing a beetroot beverage is done by mixing the beetroot powder with water to a known concentration. After drinking this beetroot beverage, the level of the nitrites/nitrates in the person's blood plasma is instantly reduced to nitric oxide. The nitrites content in the beetroot beverage is reduced by 70% within 30 minutes in the blood plasma of a human body. Further, the nitrites content in the beetroot beverage completely dissipates in few minutes. The instant reduction of the nitrite level makes the beet beverage a poor source of nitric oxide for the athletes.

Therefore, there is a need for a beet beverage composition providing controlled reduction of nitrites/nitrates to nitric oxide in the blood plasma of the human body. There is also a need for a beet beverage composition enhancing one's athletic performance and acquire other health benefits of nitric oxide for longer duration.

### SUMMARY

The present invention relates to a process of producing a beverage for enhancing athletic performance, and acquire other health benefits of nitric oxide for longer duration. In an embodiment, the process comprises selecting an amount of beet product as a solute in the first step. In one embodiment, the beet product is in the form of one or more of a beet extract, a beet concentrate, a beet powder, or a beet kibble. In an embodiment, the process of the proposed invention includes infusing water with a molecular hydrogen in a desired amount to produce hydrogen infused water as a solvent in the second step. In the final step, the process includes mixing the solute into the solvent to produce the beverage for providing controlled reduction of nitrates in a human body.

In an embodiment, the beverage comprises at least 1% of the beet product in at least 8 Oz of hydrogen infused water. In one embodiment, the solvent comprises at least 0.4 ppm of molecular hydrogen in water. In an embodiment, the temperature of the solvent is maintained in the range from 32° F. to 120° F. In one embodiment, the temperature of the solvent is maintained above 32° F. In another embodiment, the temperature of the solvent is maintained below 120° F. In an embodiment, the solute is mixed with the solvent for at least 15 seconds to produce the beet beverage.

In an embodiment, a beverage composition providing controlled reduction of nitrates in a human body. In one embodiment, the composition of the beverage comprises one or more beet products, and a hydrogen infused water. In one embodiment, the beverage composition comprises at least 1% of the beetroot product in at least 8 Oz of the hydrogen infused water. In an embodiment, the beet product is in the form of one or more of a beet extract, beet concentrate, beet powder, or a beet kibble. In one embodiment, the hydrogen infused water comprises a desired amount of molecular hydrogen in water. In another embodiment, the hydrogen infused water comprises at least 0.4 ppm of molecular hydrogen in water.

### BRIEF DESCRIPTION OF THE DRAWINGS

FIG. 1 exemplarily illustrates a flowchart for a process 10 of producing a beet beverage according to an embodiment.

### DETAILED DESCRIPTION

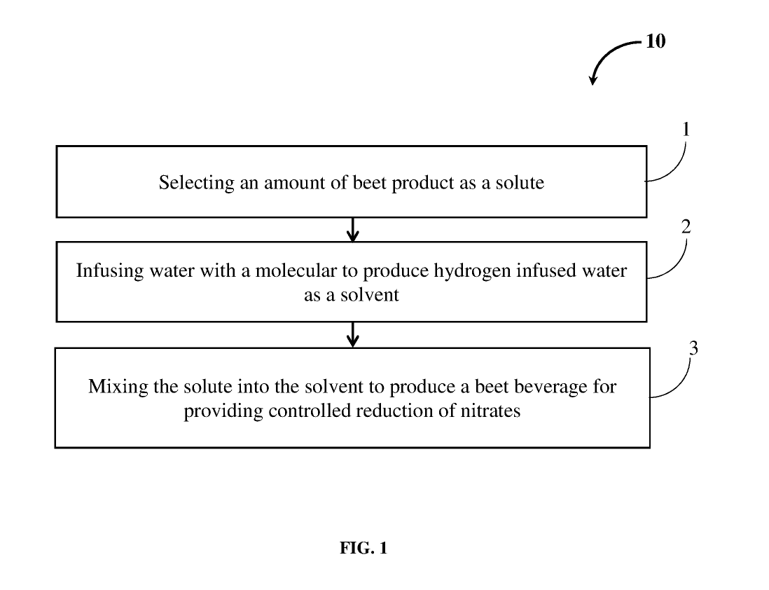

The present invention relates to a beverage composition and a process of producing the beverage using beet products. FIG. 1 exemplarily illustrates a process 10 of producing a beverage for enhancing athletic performance, and acquire other health benefits of nitric oxide for longer duration. In an embodiment, the process 10 comprises selecting an amount of beet product as a solute in step 1. In one embodiment, the beet product is in the form of one or more of a beet extract, a beet concentrate, a beet powder, or a beet kibble. In an embodiment, the process 10 of the proposed invention includes infusing water with a molecular hydrogen in a desired amount to produce hydrogen infused water as a solvent in step 2. In step 3, the process 10 includes mixing the solute into the solvent to produce the beverage for providing controlled reduction of nitrates in a human body. In an embodiment, the molecular hydrogen in the hydrogen infused water is bonded with the nitrates to enable controlled reduction of nitrates in a human body.

Referring to FIG. 1, the beverage comprises at least 1% of the beet product in at least 8 Oz of hydrogen infused water according to the process 10 of the invention. In one embodiment, the solvent comprises at least 0.4 ppm of molecular hydrogen in water according to step 2 of the invention. In an embodiment, the temperature of the solvent is maintained in the range from 32° F. to 120° F. In one embodiment, the temperature of the solvent is maintained above 32° F. In another embodiment, the temperature of the solvent is maintained below 120° F. In an embodiment, the solute is mixed with the solvent for at least 15 seconds to produce the beet beverage according to step 3 of the invention. In an embodiment, the beverage is packaged in a bottle, can, or in an aluminum container.

In an embodiment, a beverage composition providing controlled reduction of nitrates in a human body. In one embodiment, the composition of the beverage comprises one or more beet products, and a hydrogen infused water. In one embodiment, the beverage composition comprises at least 1% of the beetroot product in at least 8 Oz of the hydrogen infused water. In an embodiment, the beet product is in the form of one or more of a beet extract, beet concentrate, beet powder, or a beet kibble. In one embodiment, the hydrogen infused water comprises a desired amount of molecular hydrogen in water. In another embodiment, the hydrogen infused water comprises at least 0.4 ppm of molecular hydrogen in water.

The advantage of the proposed beverage is capable of stabilizing the nitrates/nitrites content contained in the beet beverage. The controlled reduction of nitrates for more than 3 hours to nitric oxide in the blood plasma, which is effective for enhancing one's athletic performance. The beverage also helps in acquiring other health benefits of nitric oxide for longer duration. The beet beverage could be used as a ready to drink (RTD) beverage packaged in the bottle or can.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims.

1. A process of producing a beverage, the method comprising:
a first step of selecting an amount of beet product in the form of one or more of a beet extract, a beet concentrate, a beet powder, or a beet kibble as a solute;
a second step of infusing water with a molecular hydrogen in a desired amount to produce hydrogen infused water as a solvent, and
a final step of mixing the solute into the solvent to produce the beverage for providing controlled reduction of nitrates in a human body.

2. The process of claim 1, wherein the beverage comprising at least 1% of the beet product in at least 8 Oz of hydrogen infused water.

3. The process of claim 1, wherein the solvent comprises at least 0.4 ppm of molecular hydrogen in water.

4. The process of claim 1, wherein the temperature of the solvent is maintained in the range from 32° F. to 120° F.

5. The process of claim 1, wherein the temperature of the solvent is maintained above 32° F.

6. The process of claim 1, wherein the temperature of the solvent is maintained below 120° F.

7. The process of claim 1, wherein the solute is mixed with the solvent for at least 15 seconds to produce the beet beverage.

8. A beverage composition providing controlled reduction of nitrates in a human body, comprising:
one or more beet product; and
a hydrogen infused water,
wherein the beverage composition comprises at least 1% of the beetroot product in at least 8 Oz of the hydrogen infused water.

9. The beverage composition of claim 8, wherein the beet product is in the form of one or more of a beet extract, a beet concentrate, a beet powder, or a beet kibble.

10. The beverage composition of claim 8, wherein the hydrogen infused water comprises a desired amount of molecular hydrogen in water.

11. The beverage composition of claim 8, wherein the hydrogen infused water comprises at least 0.4 ppm of molecular hydrogen in water.
